(19) United States
(12) Patent Application Publication
(10) Pub. No.: US 2021/0219821 A1
(43) Pub. Date: Jul. 22, 2021

(54) STEERABLE SHAFT FOR INTERVENTIONAL DEVICES

(71) Applicant: EnTellect Medical Holdings, Crestwood, KY (US)

(72) Inventors: Anthony Appling, Crestwood, KY (US); Ben Morris, Jeffersonville, IN (US); Brian Keith Wells, Lagrange, KY (US)

(21) Appl. No.: 17/151,842

(22) Filed: Jan. 19, 2021

Related U.S. Application Data
(60) Provisional application No. 62/961,921, filed on Jan. 16, 2020.

(51) Int. Cl. A61B 1/005; A61B 1/008

(52) U.S. Cl. CPC A61B 1/0056; A61M 25/0054; A61B 1/008; A61B 1/0057

(57) ABSTRACT

A steerable shaft for an endoscope includes a plurality of shaft segments extending about an axis and positioned in axially end-to-end and pivotable relationship with one another between a first shaft end and a second shaft end. Each of the plurality of shaft segments define at least one wire lumen, with the wire lumens disposed in axially aligned relationship with one another in a neutral position of the steerable shaft. At least one control wire extends through the aligned wire lumens between t for pivoting the plurality of shaft segments relative to one another and moving the steerable shaft from the neutral position to a deflected position in response to tensioning of the control wire. At least one spine element is connected to the plurality of shaft elements for limiting the pivoting movement of the shaft segments and related movement of the steerable shaft to an articulation plane.

### CROSS-REFERENCES TO RELATED APPLICATIONS

This application claims the benefit of U.S. Provisional Patent Application Ser. No. 62/961,921 filed on Jan. 16, 2020, the entire disclosure of which is hereby incorporated by reference in its entirety.

### TECHNICAL FIELD

The present disclosure relates to interventional devices such as those used in medical operations. More particularly, the present disclosure relates to a steerable shaft for an interventional device such as an endoscope.

### BACKGROUND OF THE DISCLOSURE

Interventional devices are used for visualizing surfaces inside objects. For example, an endoscope is a medical instrument for visualizing the interior of a patient's body. Endoscopes can be used for a variety of different diagnostic and interventional procedures, including colonoscopy, bronchoscopy, thoracoscopy, laparoscopy, ureteroscopy and video endoscopy. Endoscopes typically have a control handle which is configured to allow a user to control a position of a distal tip during the procedure to investigate for the presence of any undesirable objects, such as the presence of kidney stones, polyps or tumors during a ureteroscopy procedure. However, there remains a need for endoscopes (and other interventional devices) with improved steerable shafts disposed within, or otherwise associated with, the endoscope tube for use in adjusting the position of the distal tip.

### SUMMARY OF THE DISCLOSURE

A steerable shaft for an interventional device includes a plurality of shaft segments each extending about an axis and positioned in axially end-to-end and pivotable relationship with one another between a first shaft end and a second shaft end. Each of the plurality of shaft segments define at least one wire lumen, with the at least one wire lumen of each of the plurality of shaft segments disposed in axially aligned relationship with one another between the first and second shaft ends in a neutral position of the steerable shaft. At least one control wire extends through the aligned wire lumens between the first and second shaft ends for pivoting the plurality of shaft segments relative to one another and moving the steerable shaft from the neutral position to a deflected position in response to tensioning of the control wire. At least one spine element is connected to the plurality of shaft elements for limiting the pivoting movement of the shaft segments and related movement of the steerable shaft to an articulation plane.

The steerable shaft provides a simple and effective manner of flexing a distal tip of the interventional device along the articulation plane for controlled, consistent steering of the distal tip. Furthermore, the steerable shaft is simple in design and thus inexpensive and easy to manufacture.

### BRIEF DESCRIPTION OF THE DRAWINGS

Other aspects of the present disclosure will be readily appreciated, as the same becomes better understood by reference to the following detailed description when considered in connection with the accompanying drawings wherein:

FIG. 1 is a perspective view of an exemplary endoscope;

FIG. 2 is a perspective view of a first embodiment of a steerable shaft illustrating a plurality of shaft segments interconnected from a first shaft end to a second shaft end;

FIG. 3 is a rear, perspective view of the first embodiment of the steerable shaft disposed in a deflected condition;

FIG. 4 is a perspective view of a distal segment of the plurality of shaft segments of the first embodiment of the steerable shaft illustrating a control wire passing through a pair of wire lumens and across a cleat for securing the control wire to the distal segment;

FIG. 5 is an exploded perspective view of a second embodiment of the steerable shaft disposed in a neutral condition and illustrating a pair of spine elements disposed on opposite sides of the steerable shaft for mating with respective wire slots to maintain deflection of the steerable shaft along a single articulation plane;

FIG. 6 is a partial perspective view of the second embodiment of the steerable shaft deflected along the articulation plane;

FIG. 7 is a perspective view of one of a plurality of shaft segments of the second embodiment of the steerable shaft illustrating the wire slots extending axially along an outer circumference of the segment body;

FIG. 8 is an end view of one of the plurality of shaft segments of the second embodiment of the steerable shaft illustrating the pair of wire lumens and a central lumen defined by the segment body for receiving instruments such as electronics wiring or catheter components;

FIG. 9 is a perspective view of a third embodiment of the steerable shaft deflected along the articulation plane and illustrating protruding and receiving components of spine elements for providing deflection of the steerable shaft along the articulation plane;

FIG. 10 is a perspective view of one of a plurality of shaft segments of the third embodiment of the steerable shaft illustrating the protruding and receiving components and a wire lumen;

FIG. 11 is a top view of one of the plurality of shaft segments of the third embodiment of the steerable shaft;

FIG. 12 an end view of one of the plurality of shaft segments of the third embodiment of the steerable shaft;

FIG. 13 is a perspective view of a fourth embodiment of the steerable shaft illustrating protruding and receiving components of a plurality of shaft segments for providing deflection of the steerable shaft along the articulation plane and illustrating axially aligned slots which define wire lumens;

FIG. 14 is a perspective view of the fourth embodiment of the steerable shaft illustrating the steerable shaft connected to an endoscope tube and illustrating a window defined by the endoscope tube through which a control wires passes;

FIG. 15 is a perspective view of a fifth embodiment of the steerable shaft illustrating an alternate arrangement of protruding and receiving components of a spine element for providing deflection of the steerable shaft along the articulation plane;

FIG. 16 is a perspective view of one of a plurality of shaft segments of the fifth embodiment of the steerable shaft;

FIG. 17 is a top view of two of the shaft segments of the fifth embodiment of the steerable shaft illustrating coupling of the shaft segments along the protruding and receiving components;

FIG. 18 is a fragmentary top view of one of the shaft segments of the fifth embodiment of the steerable shaft;

FIG. 19 is a side view of one of the shaft segments of the fifth embodiment of the steerable shaft;

FIG. 20 is a top view of the fifth embodiment of the steerable shaft illustrating laser cutting patterns that may be used to create the steerable shaft;

FIG. 21 is a perspective of a sixth embodiment of the steerable shaft illustrating a monolithic tube extending along an axis from the first shaft end to the second shaft end and illustrating a plurality of slots and ribs that provide pivoting of the steerable shaft along the articulation plane;

FIG. 22 is a second shaft end view of the sixth embodiment of the steerable shaft;

FIG. 23 is a perspective view of the sixth embodiment of the steerable shaft illustrating alternating ones of the plurality of ribs bent radially towards the axis to establish a pathway of wire lumens for receiving the control wires;

FIG. 24 is a second shaft end view of the sixth embodiment of the steerable shaft illustrating the control wires passing through the pathway defined by the plurality of ribs and slots;

FIG. 25 is a perspective view of the sixth embodiment of the steerable shaft illustrating an alternative arrangement of securing the control wire to the monolithic tube via passing the control wire through a cleat disposed adjacent the second shaft end;

FIG. 26 is a top view of a portion of FIG. 25 more clearly illustrating the cleat disposed adjacent the second shaft end and with the control wire removed;

FIG. 27 is a second shaft end view of the sixth embodiment of the steerable shaft illustrating the cleat;

FIG. 28 is a second shaft end view of the sixth embodiment of the steerable shaft illustrating alternative arrangements of securing multiple control wire via cleats;

FIG. 29 is a top view of a seventh embodiment of the steerable shaft disposed in a neutral position and illustrating a plurality of wave washer shaped shaft segments stacked on one another and interconnected from the first shaft end to the second shaft end;

FIG. 30 is a top view of the seventh embodiment of the steerable shaft disposed in a deflected position;

FIG. 31 is an end view of one of the plurality of wave washer shaped shaft segments of the seventh embodiment in an initially formed flat pattern and defining a pair of opposing wire slots, a pair of opposing wire lumens, and a central lumen;

FIG. 32 is a top view of the shaft segment of FIG. 31;

FIG. 33 is a perspective view of the shaft segment of FIG. 31 in a final formed shape and defining a cup portion disposed between the pair of opposing wire lumens;

FIG. 34 is a top view of the shaft segment of FIG. 33 in the final formed shape;

FIG. 35 is an end view of one of a plurality of shaft segments of an alternate arrangement of the seventh embodiment of the steerable shaft in an initially formed flat pattern to define a plurality of wire slots, a plurality of wire lumens, and a pair of central lumens;

FIG. 36 is a top view of the shaft segment of FIG. 35;

FIG. 37 is a top view of the shaft segment of FIG. 35 in a secondary formed shape and defining a pair of cup portions disposed on opposite sides of a central crest portion;

FIG. 38 is a perspective view of the alternative arrangement of the shaft segment of FIG. 35 in a final formed shape and folded along the central crest portion to dispose the plurality of wire slots, the plurality of wire lumens, the pair of central lumens, and the pair of cup portions in aligned relationship with one another; and

FIG. 39 is a top view of the shaft segment of FIG. 35.

### DETAILED DESCRIPTION OF THE EXEMPLARY EMBODIMENTS

In the following description, details are set forth to provide an understanding of the present disclosure. In some instances, certain systems, structures and techniques have not been described or shown in detail in order not to obscure the disclosure.

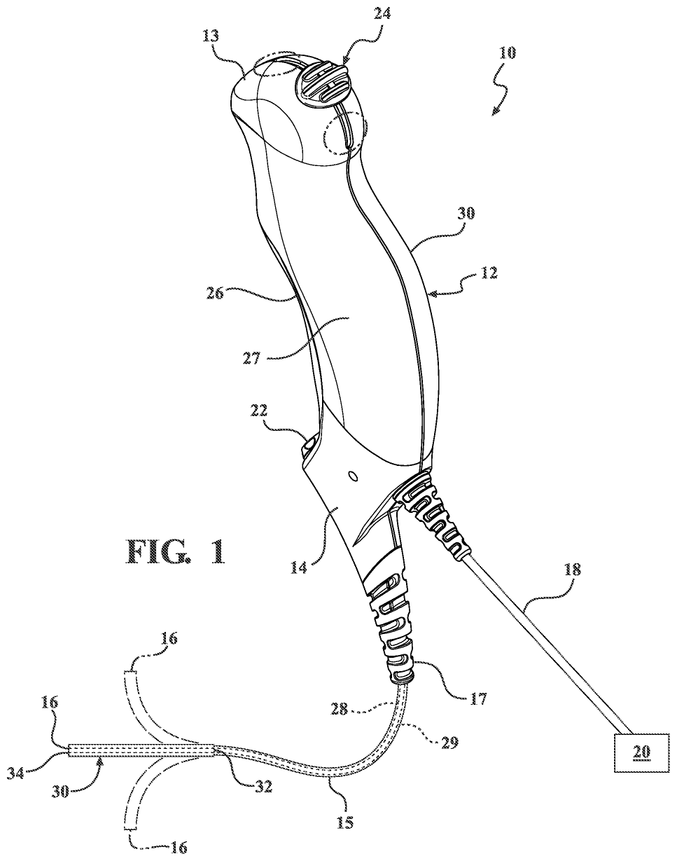

Referring to the Figures, wherein like numerals indicate corresponding parts throughout the several views, an interventional device 10 is generally shown. According to the example embodiment, the interventional device is an endoscope 10, however, the teachings herein may be applied to other types of interventional devices 10. Moreover, the endoscope 10 shown in the figures could be utilized in association with various diagnostic and interventional procedures, such as ureteroscopy procedures, without departing from the scope of the subject disclosure. As illustrated in FIG. 1, the endoscope 10 includes a control handle 12 extending from a proximal end 13 to a distal end 14. An endoscope tube 15 extends from the control handle 12, adjacent the distal end 14, and terminates at a distal tip 16 for being located inside a patient's body for diagnostic and interventional procedures, such as the identification of kidney stones, polyps or tumors in the case of ureteroscopy. A strain relief 17 surrounds the endoscope tube 15 at an interface of the endoscope tube 15 and the control handle 12 to provide flexibility to, and for protecting the endoscope tube 15. An umbilical cable 18 extends from the control handle 12 for being coupled with a processing device 20 for evaluating data obtained by the distal tip 16. The processing device 20 may include various types of processors that may be configured to display image data captured by the distal tip 16 or perform various other analytical functions. A port 22 is located on the control handle 12 that provides access to the working channel of the endoscope tube 15 located between the port 22 and the distal tip 16. The port 22 allows attachment of user prescribed accessories and provides access for insertion of tools and irrigation.

With further reference to FIG. 1, a steering assembly 24 is provided on the control handle 12 for allowing a user to control movement of the distal tip 16 of the endoscope tube 15 during the diagnostic and interventional procedure. A gripping region 26 is located between the proximal and distal ends 13, 14 of the control handle 12 and is shaped to receive a palm and fingers of a user to provide easy, comfortable gripping of the control handle 12 by the user during use of the steering assembly. The control handle 12 is preferably comprised of a first shell 27 and a second shell 30 that are coupled with one another to define a hollow compartment. However, the control handle 12 could also be a unitary component without departing from the scope of the subject disclosure.

As illustrated throughout the Figures, at least one control wire (or cable) 28, 29 extends from the compartment of the control handle 12, through the distal end 14, into the endoscope tube 15 and ultimately terminates adjacent the distal tip 16. The at least one control wire 28, 29 is configured to deflect the distal tip 16 from a neutral position to a deflected position in response to rotational movement of the steering assembly 24 which applies tension to the at least one control wire 28, 29 depending on a directional of movement of the steering assembly 24. In a preferred arrangement shown in FIG. 1, the at least one control wire 28, 29 includes a first control wire and a second control wire 29 configured to deflect the distal tip 16 from the neutral position (shown in solid lines) to two opposite deflected positions (illustrated in broken lines) in response to rotational movement of the steering assembly 24 in two different rotational directions (illustrated in broken lines).

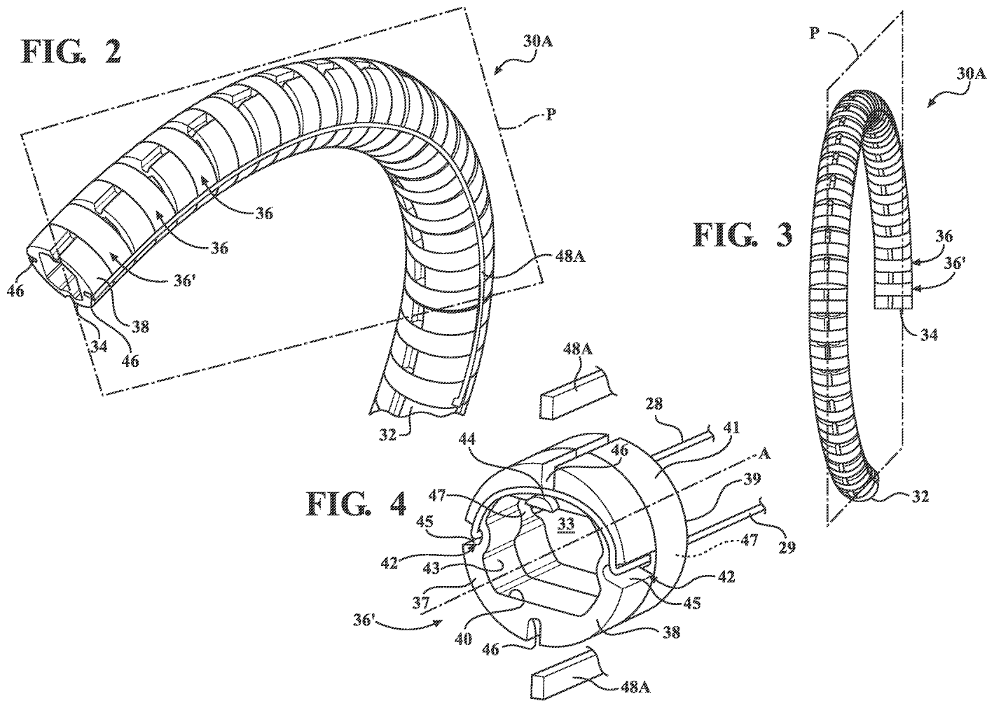

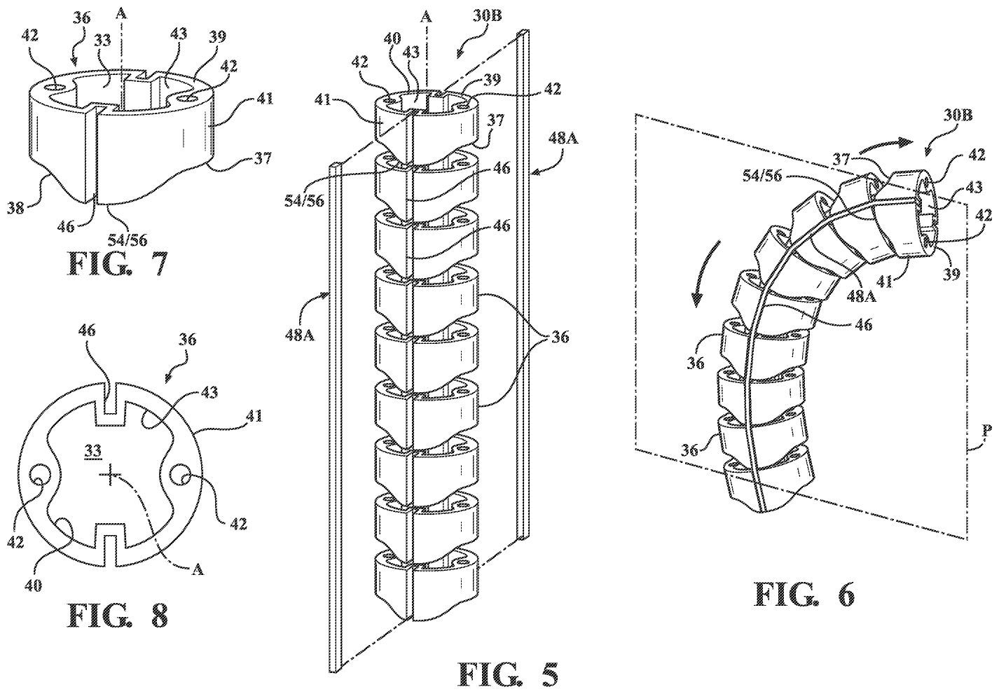

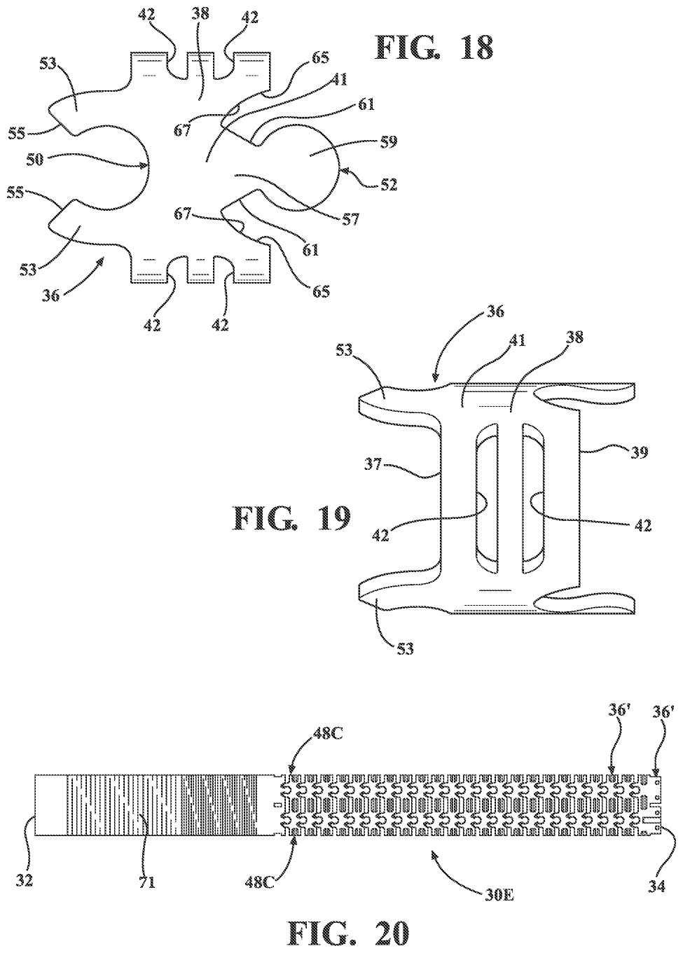

As mentioned previously, a position of the distal tip 16 of the endoscope 10 is adjusted during the diagnostic and interventional procedure to investigate for the presence of any undesirable objects, such as the presence of kidney stones, polyps or tumors during an ureteroscopy procedure. Accordingly, as best illustrated in FIGS. 2-3, 7-9, 13-15, 17, 19-20 and 23-24, the endoscope 10 includes a steerable shaft 30-30F disposed within, or otherwise associated with, the endoscope tube 15. For example, as illustrated in FIG. 1, the steerable shaft 30-30F may extend along any length of the endoscope tube 15, but according to the example embodiment, extends a predetermined length away from the distal tip 16 such that it may be used in adjusting the position of the distal tip 16. The steerable shaft 30-30F extends from a first shaft end 32 to a second shaft end 34 disposed proximate to the distal tip 16. As illustrated by FIGS. 4, 13-14, 7-19 and 21-22, the at least one control wire 28, 29 extends along the steerable shaft 30-30F and is secured adjacent the second shaft end 34 for use in adjusting the steerable shaft 30-30F from a neutral position (such as shown in FIGS. 5, 13-15, 17, 21, 23 and 29) to a deflected position (such as shown in FIGS. 2, 3, 6, 9, 19, 24 and 30). As will be discussed in further detail below, the steerable shaft 30A-30G includes a spine element 48A-48D that limits pivoting of the steerable shaft 30A-40G to or along a single articulation plane P during movement between the neutral and deflected positions to correspondingly adjust the position of the distal tip 16 of the endoscope tube 15.

With reference to FIGS. 2-4, in accordance with a first embodiment, the steerable shaft 30A includes a plurality of individual shaft segments 36 interconnected with one another between the first and second shaft ends 32, 34. Each of these individual shaft segments 36 could be molded, machined or have an extruded profile that is laser cut into segments (See, e.g. FIG. 10-20). The shaft segments 36 are preferably comprised of metal or polymer but could be comprised of other materials without departing from the scope of the subject disclosure. As best illustrated in FIG. 4, each of the shaft segments 36 include a segment body 38 which defines a central lumen 40 passing therethrough. Each of the shaft segments 36 extends axially between a first axial end 37 and a second axial end 39 to define a radially outer surface 41 and a radially inner surface 43. As illustrated in FIGS. 2-3, when the individual shaft segments 36 are coupled with one another, the central lumens 40 of the interconnected shaft segments 36 are disposed in aligned relationship to define a central passageway 33 passing through the endoscope tube 15 and terminating at the distal tip 16 for receiving instruments, such as electronics wiring or catheter components.

As best illustrated in FIG. 4, each of the segment bodies 38 defines a pair of wire lumens 42 disposed on diametrically opposite sides of the central lumen 40 in aligned relationship with one another. According to this embodiment, each of the wire lumens 42 includes a first wire segment 47 configured as a trough extending axially along the radially inner surface 43, and a second wire segment 45 configured as a trough extending axially along the radially outer surface 41, with the first and second wire segments 47, 45 axially aligned with one another such that the wire lumen 42 provides radial support to the control wire 28, 29 in both radial directions while still providing access to the control wire 28, 29. The at least one control wire 28, 29 passes serially through all of the aligned wire lumens 42 and ultimately terminates at, or wraps around, a distal segment 36′ of the plurality of shaft segments 36 disposed adjacent the shaft end 34. According to an embodiment, the at least one control wire 28, 29 includes two separate control wires 28, 29, with each control wire 28, 29 terminating and coupled at the shaft end 34. Alternatively, in a preferred arrangement best illustrated in FIG. 4, the distal segment 36′ includes a cleat 44, and the first and second control wires 28, 29 are integrally connected to one another to effectively define a single control wire 28, 29 which is looped around the cleat 44 to establish secured relationship with the distal segment 36′.

As shown in FIGS. 2-4, each of the shaft segments 36 also preferably defines at least one wire slot 46 extending linearly/axially along an outer circumference of the segment body 38, on opposite sides of the central lumen 40 and disposed between the pair of wire lumens 42. According to the example embodiment, the at least one wire slot 46 includes a pair of wire slots 46 located on diametrically opposite sides of the shaft segment 46. When the steerable shaft 30 is disposed in the neutral position, all of the wire slots 46 are disposed in aligned relationship with one another, and the at least one spine element 48A is positioned in the aligned wire slots 46 between the first and second ends 32, 34. According to the example embodiment, the at least one spine element 48A includes a pair of spine elements 48A disposed on opposite sides of the steerable shaft within a respective one of the aligned wire slots 46. As illustrated by FIG. 2, the pair of spine elements 48A maintain deflection of the steerable shaft along the predetermined, single articulation plane P, by resisting bending of the steerable shaft 30 out of this desired plane P. According to the first embodiment, the at least one spine element 48A is an elongated wire with a rectangular cross-section, however, other arrangements of spine elements could be used without departing from the scope of the subject disclosure. Furthermore, in a preferred arrangement, each of the spine elements 48 are only joined to the distal segment of the shaft segments 36. However, the spine elements 48 could be joined or secured to additional shaft segments 36 without departing from the scope of the subject disclosure.

A second embodiment of the steerable shaft 30B is shown in FIGS. 5-8. The second embodiment is substantially the same as the first embodiment of the steerable shaft 30A, except each of the shaft segments 36 includes a pair of axially extending protrusion portions 54 protruding from the first axial end 37 of the shaft segment 36 in aligned relationship with the wire slots 46 to define a pivot arc surface 56 upon which adjacent shaft segments 36 pivot, which establishes a pivot angle during deflection of the steerable shaft 30. (See, e.g., FIG. 6). The shaft segments 36 with the pivot arc surfaces 56 provide greater articulation for the steerable shaft 30 and also further assist in keeping deflection on plane. Furthermore, according to this embodiment, the wire lumens 42 are each an axially extending channel along their lengths, rather than being defined by the radial first and second segments 47, 45.

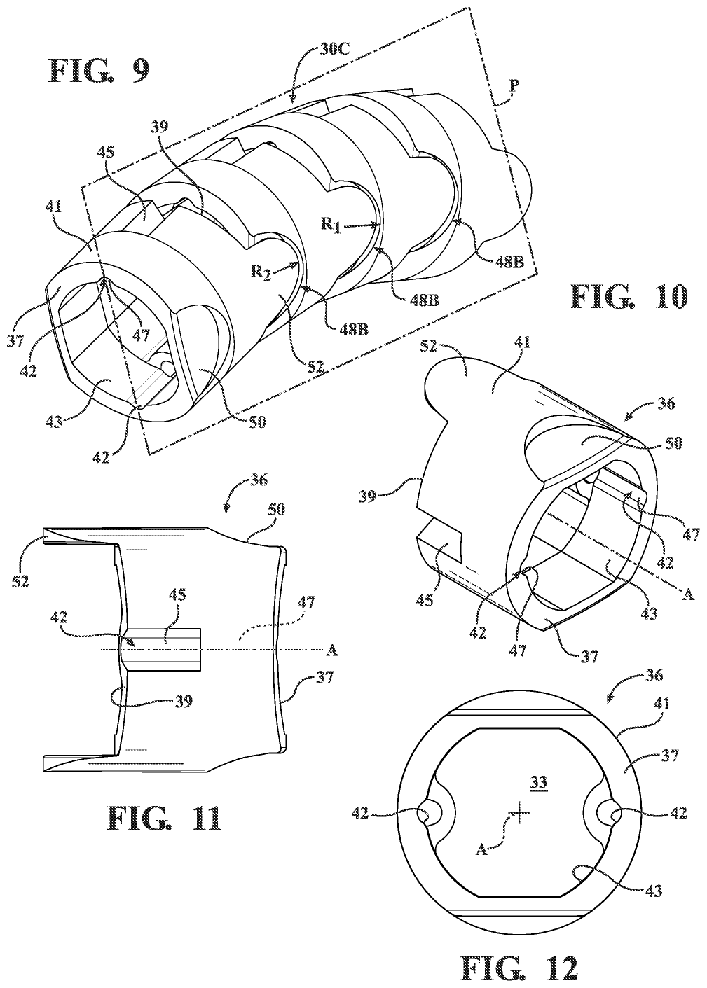

A third embodiment of the steerable shaft 30C is shown in FIGS. 9-12. According to this embodiment, rather than being comprised of the pair of flat wires 48A, the spine elements 48B are each comprised of corresponding receiving and protruding portions 50, 52 of adjacent shaft segments 36 mating with one another and pivotable relative to one another to establish the deflection on the articulation plane P. More particularly, the protruding portions 52 of each of the shaft segments 36 extend from the second axial end 39 of the shaft segment 36 and are located on diametrically opposite sides of the shaft segments 36 from one another. The receiving portions 50 of each of the shaft segments 36 are defined along the radially outer surface 41 of the shaft segment and border the first axial end 37 of the shaft segment 36 on diametrically opposite sides of the shaft segment 36 from one another. As shown in FIG. 9, each of the protruding portions 52 has an arc-shape with a first radius of curvature R1, and each of the receiving portions 50 has an arc-shape with a second radius of curvature R2. The second radius of curvature R2 is larger than the first radius of curvature R1 such that the protruding portion 52 may pivot within the receiving portion 50 along the articulation plane P. The radii of curvature R1, R2 may be varied to provide different degrees of pivoting. Each of the shaft segments 36 also includes a pair of wire lumens 42 on diametrically opposite sides of the steerable shaft 36 from one another. Furthermore, a control wire 28 extends through each of the wire lumens 42. Like the first embodiment of the steering assembly 30A, each of the wire lumens 42 includes a first wire segment 47 defined as a trough along the radially inner surface 43 of the shaft segment and a second wire segment 49 defined as a trough along the radially outer surface 41 of the shaft segment, with the first and second segments 47, 49 being axially aligned with one another. According to this embodiment (and the others) each of the shaft segments 36 could be laser cut from a metal tube or an extruded profile.

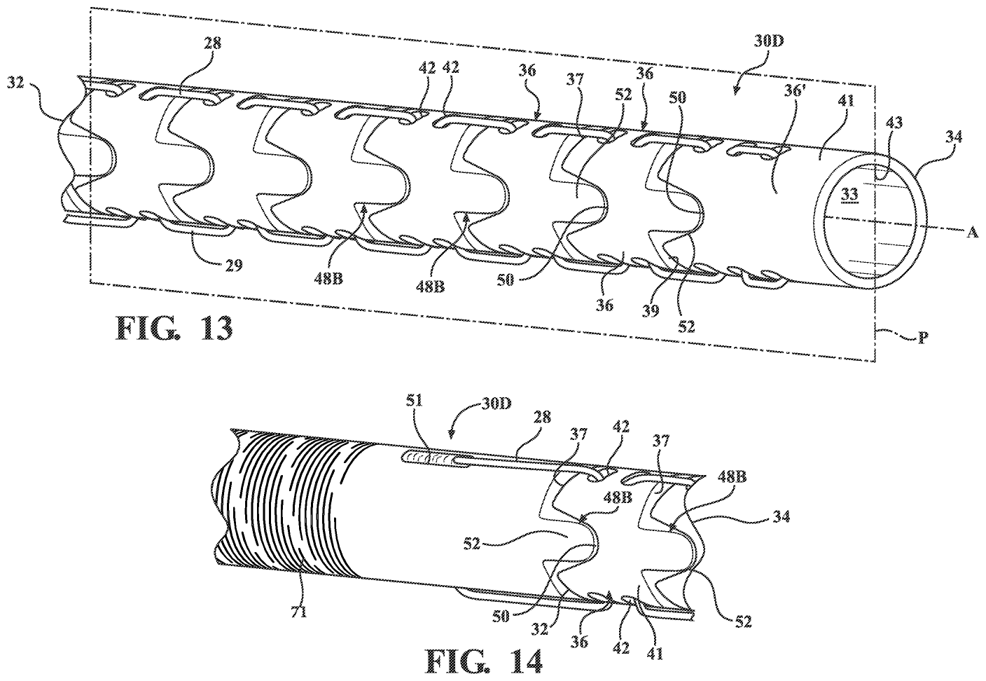

A fourth embodiment of the steerable shaft 30D is shown in FIGS. 13-14. The fourth embodiment is similar to the third embodiment of the steering assembly 30C, but the wire lumens 42 thereof are defined by a plurality of circumferentially extending slots 42 that are spaced axially from one another along each of the shaft segments 36. Two axial rows of the slots 42 are located on circumferentially opposite sides of the steerable shaft 36 as one another. As shown, a pair of control wires 28, 29 pass through and extend along each row of slots 42. Additionally, as shown, the steerable shaft 30D may also include a spring portion 71 which is at least partially comprised of a spring for deflection in all directions. As shown, an end of the spring portion 71 includes a protruding portion 52 for being received by and connected to a receiving portion 50 of one of the shaft segments 36 in the same manner that the shaft segments 36 are coupled to one another. Furthermore, FIG. 14 illustrates that a window 51 may be defined by the spring portion 71 adjacent to the shaft segments 36 through which the control wires 28, 29 may pass.

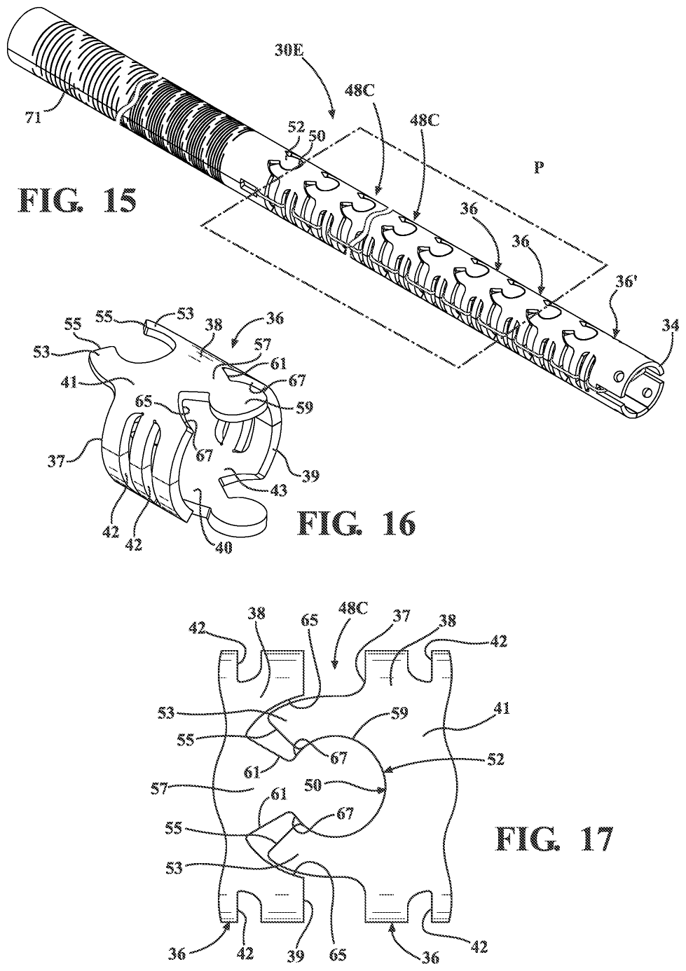

A fifth embodiment of the steerable shaft 30E is shown in FIGS. 15-20. The fifth embodiment is similar to the third and fourth embodiments, but includes additional features associated with the spine elements 48C. More particularly, according to this embodiment, the receiving portions 50 are each defined by a pairs of legs 53 that extend from the first axial end 37 of each shaft segment 36, on circumferentially opposite sides of the shaft segment 36 as one another to define two receiving portions 50. Each leg 53 extends in an arc shape from the first axial end 37 of the shaft segment 36 toward the other leg 53 to define a semi-circular shape of each receiving portion 50. Furthermore, each of the legs 53 terminates at a flat contact surface 55. The protruding portions 52 each have a neck portion 57 and a disc portion 59, with the neck portion 57 extending from the second axial end 39 and defined by two angled surfaces 61 extending generally toward one another and terminating at the disc portion 59. The disc portion 59 is sized such that it has a slightly smaller diameter than the receiving portion 50 such that the protruding portion 52 is pivotable within the receiving portion 50 to provide the pivoting movement of the shaft segments 36 relative to one another along the articulation plane P (illustrated in FIG. 15). The second axial end 39 of each shaft segments 36 further defines a pair of arc-shaped walls 65 that each extend from one of the angled surfaces 61 of the neck portion 57 to define a pair of arc-shaped channels 67 that each receive one of the legs 53 of the receiving portion 50 of another of the shaft segments 36. The pivoting movement of the shaft segments 36 relative to one another is limited in both directions by engagement of the contact surfaces 55 of the legs 53 against the neck portion 57 of the protruding portion 52.

Like the fourth embodiment, the wire lumens 42 are defined by a plurality of circumferentially extending slots 42 that are spaced axially from one another on each of the shaft segments 36, with two rows of the slots 42 located on circumferentially opposite sides of the shaft segment 36 circumferentially between the protruding and receiving portions 52, 50. As illustrated in FIG. 15, the first and second control wires 38 each extend through one of the rows of slots 42. Like previous embodiments, the first and second wires 38, 40 can be secured to the distal shaft segment 36′ or can be integrally connected to one another along the distal shaft segment 36′.

As further shown in FIG. 15, the steerable shaft 30E may also include a spring portion 71 which is at least partially comprised of a spring for deflection in all directions. As shown, an end of the spring portion 71 includes a protruding portion 52 for being received by and connected to a receiving portion 50 of one of the shaft segments 36 in the same manner that the shaft segments 36 are coupled with one another.

FIG. 20 illustrates laser cutting patterns that may be used to create the fifth embodiment of the steerable shaft 30E.

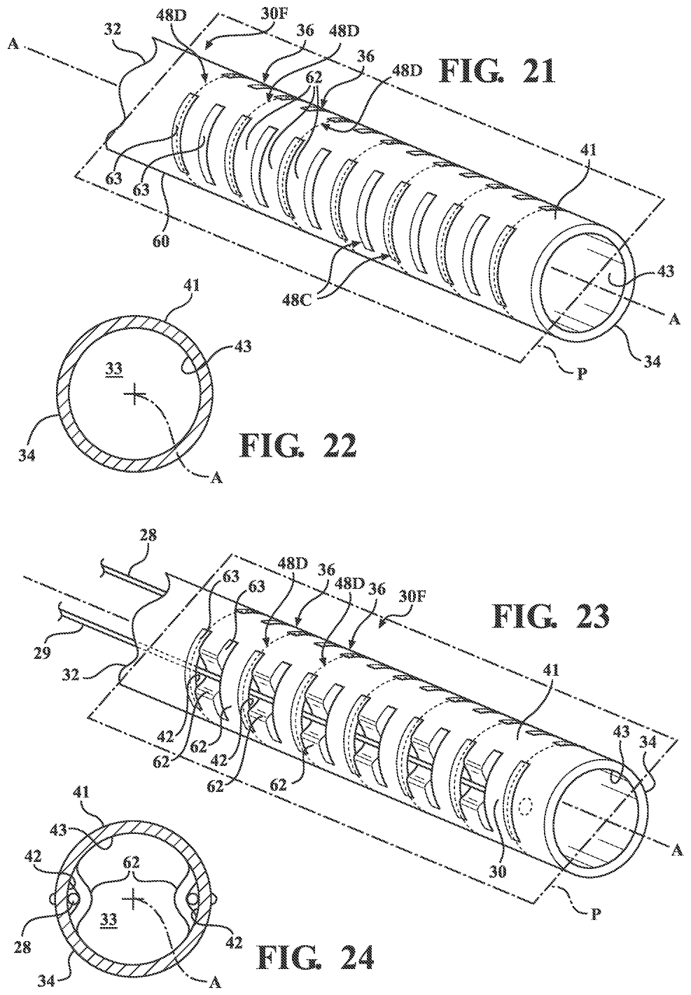

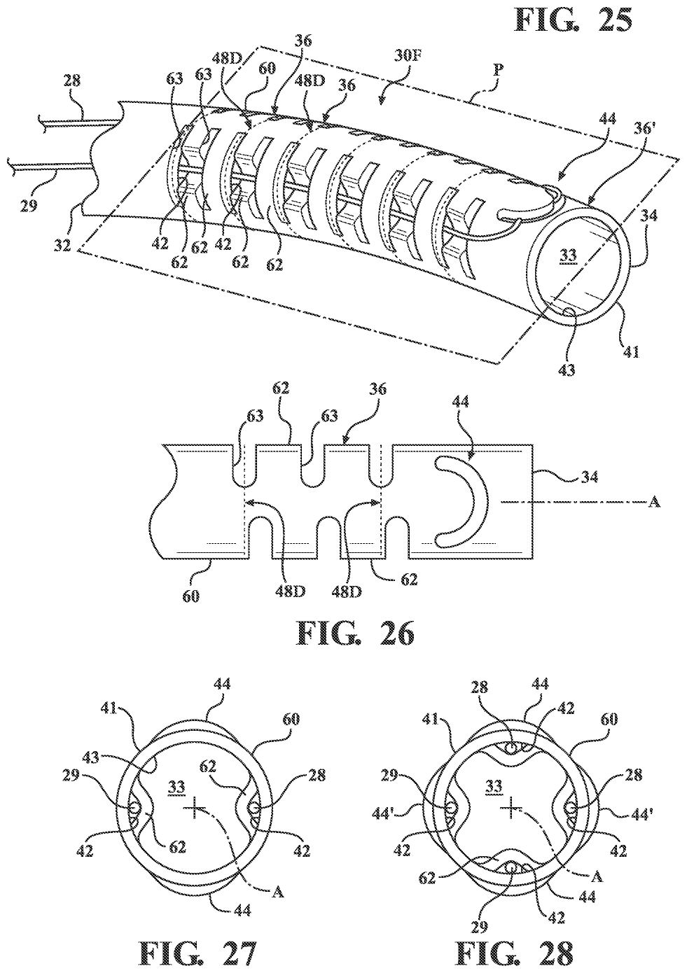

With reference to FIGS. 21-28, in accordance with a sixth embodiment, the steerable shaft 30F is arranged as an elongated tube of a relatively stiff material which defines a plurality of slots 63 that each extend circumferentially and are arranged in axially spaced relationship with one another for facilitating deflection of the steerable shaft 30F along the articulation plane P. A plurality of ribs 62 are defined between pairs of the slots 63 and arranged in axially spaced relationship with one another between the first and second axial ends 37, 39. The elongated tube is segmented into a plurality of shaft segments 36 (segments shown by broken lines in FIGS. 21, 23 and 25) that are each defined by the plurality of the slots 63. According to the example embodiment, each of the shaft segments 36 includes three slots 63 and two ribs 62, however, other numbers of slots 63 and corresponding ribs 62 could be encompassed by each shaft segment 36. As best illustrated in FIGS. 23 and 25, alternating ones of the plurality of ribs 62 are bent radially towards the axis A to establish a pathway of wire lumens/eyelets 42 for receiving the at least one control wire 28, 29 which extends along an inner diameter of the monolithic tube 60. As illustrated in FIG. 25, an arrangement of the control wires 28, 29 in the wire lumens 42 defined by the slots 63 and ribs 62 provide deflection of the steerable shaft 30F along the single articulation plane P.

According to a further aspect of the sixth embodiment, the spine elements 48D are comprised of integral connection 48C of each of the shaft segments 36 at locations of the shaft segments 36 that are circumferentially out of alignment with the slots 63 and the ribs 62. The integral connections 48D are of a relatively stiff material that resists pivoting of the steerable shaft 30F in directions transverse to the articulation plane P in order to limit pivoting of the steerable shaft 30F to the articulation plane P.

As illustrated in FIGS. 23, in one arrangement of the sixth embodiment, the at least one control wire 28, 29 can include a pair of control wires 28, 29 extending along opposite sides of the inner diameter, with each being secured to the steerable shaft 30F adjacent the second shaft end 34. According to another arrangement shown in FIG. 25, the control wires 28, 29 are integrally connected to constitute a single control wire 28, 29 which passes through the inner diameter and is hooked or looped across a cleat 44 to secure the single wire 28, 30 to the monolithic tube 60. As illustrated in FIG. 27, the cleat feature can include two cleats 44 to define a bi-directional single plane articulation, or as shown in FIG. 28, the cleat feature can include four cleats 44, 44′ disposed in 90° relationship to one another to define a 4-way direction, dual plane articulation. Although not expressly shown, in the fifth embodiment, the plurality of ribs 62 can be of varying widths to provide for different flexibility and could even have interlocking shape cuts or other combinations.

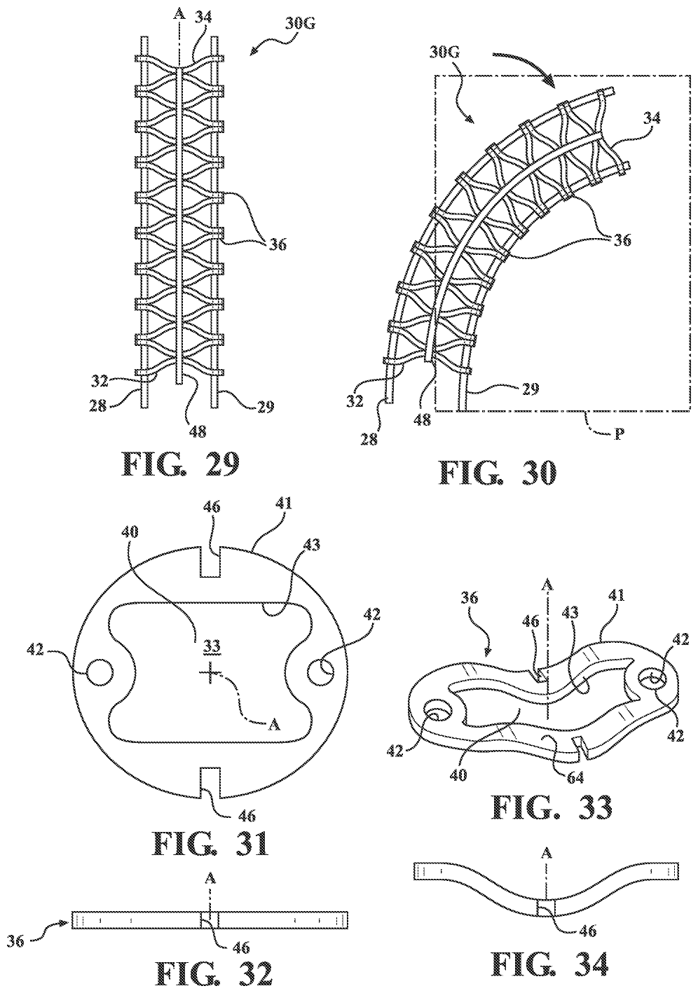

With reference to FIGS. 29-39, in accordance with a seventh embodiment of the steerable shaft 30G, the shaft segments 36 are comprised of a plurality of wave washer shaped segments 36 which are stacked on one another along the axis A between the first shaft end 32 and the second shaft end 34 to form the steerable shaft 30. As illustrated in FIGS. 31-32, in a preferred arrangement of manufacturing, each of the wave washer shaped segments 36 are first stamped, formed, or otherwise fabricated, such as by photoetching a sheeting, to initially form a flat pattern for each of the segments 36. Similar to the first embodiment, each of the segments 36 include a pair of opposing wire lumens 42, a pair of opposing wire slots 46, and a central lumen 40. As illustrated in FIGS. 33-34, each of the flat pattern segments 36 are then secondarily die stamped or otherwise formed to define a final formed shape for the wave washer shaped segments 36 which includes a cup portion 64/protrusion portion 64 disposed between the pair of wire slots 46. As best illustrated in FIGS. 29-30, each of the segments 36 are then stacked on one another between the first and second shaft ends 32, 34, in alternating arrangement of the cup portions 64, with adjacent cup portions 64 extending axially toward one another and engaging one another. A pair of control wires 28 pass through the wire lumens 42 to secure the segments 36 and form the steerable shaft 30. As further illustrated in FIGS. 29-30, a pair of spine elements/flat wires 48 (like those of the first and second embodiments of the steerable shaft 30A, 30B) are disposed along the aligned wire slots 46, with preferred attachment to a distal segment of the stacked segments 36, to maintain axial alignment of the segments 36 and as well as deflection of the steerable shaft 30 along the single articulation plane P.

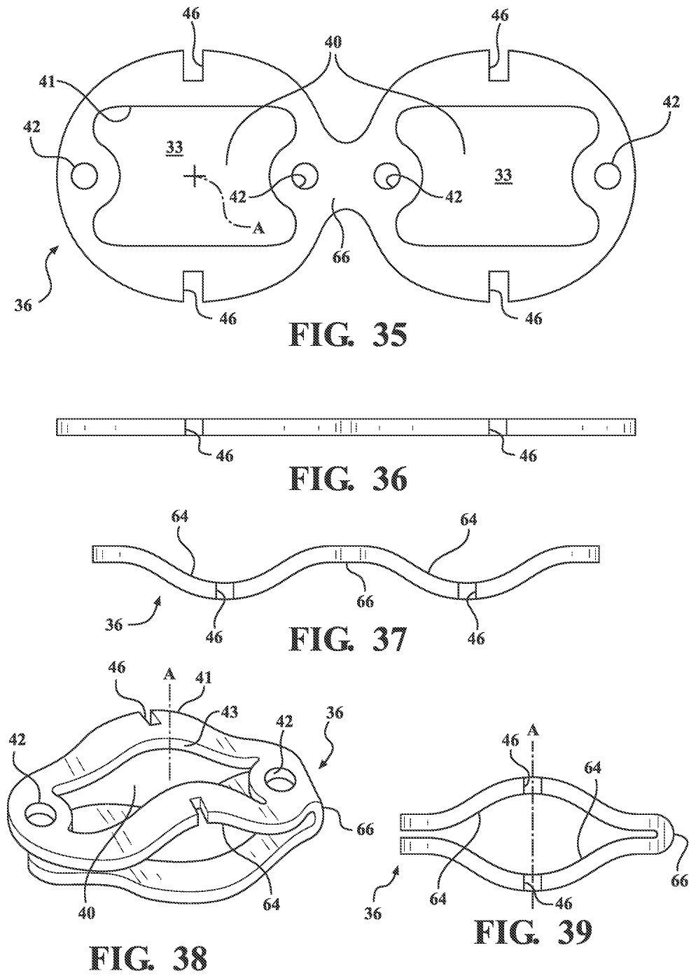

As illustrated in FIGS. 35-39, in accordance with another aspect of the seventh embodiment, each of the initially formed flat patterns of the segments 36 can include a pair of shaft segments 36 which are mirror images of one another (i.e., a dual piece segment). During the secondary forming processing, a central crest portion (edge portion) 66 is formed between the mirror images which integrally connects the segments 36, and then each shaft segment 36 is folded along the central crest portion 66 to define the wire lumens 42, the wire slots 46 and the central lumens 40 in aligned relationship with one another to form opposing, but integral, dual shaft segments 36. These dual shaft segments 36 are then stacked on one another between the first and second shaft ends 32, 34 to form the steerable shaft 30 with adjacent cup portions 64 extending axially toward and engaging one another. In either arrangement, a height (formed radius) of the cup portions 64 can be varied on different segments 36 over a length of the steerable shaft to affect the amount of flexure at that point.

Obviously, many modifications and variations of the present disclosure are possible in light of the above teachings and may be practiced otherwise than as specifically described.

What is claimed is:

1. A steerable shaft for an interventional device, comprising:
a plurality of shaft segments each extending about an axis and positioned in axially end-to-end and pivotable relationship with one another between a first shaft end and a second shaft end;
each of the plurality of shaft segments defining at least one wire lumen, with the at least one wire lumen of each of the plurality of shaft segments being disposed in axially aligned relationship with one another between the first and second shaft ends in a neutral position of the steerable shaft;
at least one control wire extending through the aligned wire lumens between the first and second shaft ends for pivoting the plurality of shaft segments relative to one another and moving the steerable shaft from the neutral position to a deflected position in response to tensioning of the control wire; and
at least one spine element connected to the plurality of shaft elements for limiting the pivoting movement of the shaft segments and related movement of the steerable shaft to an articulation plane.

2. The steerable shaft for an interventional device as set forth in claim 1, wherein the at least one spine element includes a pair of spine elements disposed on diametrically opposite sides of the plurality of shaft elements.

3. The steerable shaft for an interventional device as set forth in claim 2, wherein each of the shaft segments extend between a first axial end and a second axial end, wherein the pairs of spine elements includes a pair of receiving portions defined at the first axial end of each of the shaft segments on circumferentially opposite sides of the shaft segments, and a pair of protruding portions extending from the second axial end of each of the shaft segments and each received by one of the receiving portions of another of the shaft segments and pivotable at the receiving portions along the articulation plane.

4. The steerable shaft for an interventional device as set forth in claim 3, wherein each of the protruding portions has an arc-shape to define a first radius of curvature, and each of the receiving portions has an arc-shape to define a second radius of curvature being larger than the first radius of curvature for allowing the protruding portion to pivot within the receiving portion along the articulation plane.

5. The steerable shaft for an interventional device as set forth in claim 3, wherein the receiving portions are each defined by a pair of legs that each extend from the first axial end of the shaft segment in an arc-shape in converging relationship with the other of the pair of legs to terminate at an end surface disposed in spaced relationship with the end surface of the other of the pair of legs to define a semi-circular shape of each receiving portion between the pair of legs, wherein the second axial end of each of the shaft segments defines a pair of arc-shaped channels about the protrusion portion that each terminate at a neck portion of the protrusion portion, wherein the pair of legs are each received in one of the channels, and wherein pivoting of the shaft segments relative to one another is limited by engagement of the end surfaces of the legs with the neck portions in the arc-shaped channels.

6. The steerable shaft for an interventional device as set forth in claim 1, wherein each of the shaft segments has a radially outer surface and a radially inner surface, and wherein the at least one wire lumen includes a first wire segment shaped as a trough along the radially inner surface of the shaft segment, and a second wire segment shaped as a trough along the radially outer surface of the shaft segment, and wherein the first and second wire segments are axially aligned with one another.

7. The steerable shaft for an interventional device as set forth in claim 1, wherein each of the shaft segments defines a first set of wire lumens extending circumferentially and axially spaced from one another, a second set of wire lumens extending circumferentially and axially spaced from one another, wherein the first and second sets of wire lumens are located on diametrically opposite sides of the shaft segment from one another, each in circumferentially spaced relationship with the at least one spine element, and wherein the at least one control wire includes a pair of control wires each passing through one of the sets of wire lumens.

8. The steerable shaft for an interventional device as set forth in claim 1, wherein the at least one wire lumen includes a first wire lumen and a second wire lumen on diametrically opposite sides of the steerable shaft from one another, each in circumferentially spaced relationship with the at least one spine element, wherein the first wire lumen and the second wire lumen each extend axially between the first and second axial ends, and wherein the at least one control wire includes a first control wire extending through the first wire lumens of the plurality of shaft segments and a second control wire extending through the second wire lumens of the plurality of shaft segments.

9. The steerable shaft for an interventional device as set forth in claim 8, wherein the first control wire and the second control wire are each fixed to one of the shaft segments at the second shaft end.

10. The steerable shaft for an interventional device as set forth in claim 8, wherein the first control wire and the second control wire are integrally connected to one another adjacent to the second shaft end to constitute a single control wire.

11. The steerable shaft for an interventional device as set forth in claim 1, wherein a radially outer surface of each of the shaft segments defines at least one wire slot, wherein the wire slots of the shaft segments are aligned relationship with one another to collectively define a wire channel, and wherein the at least one spine element includes at least one flat wire received in the wire channel and bendable only along the articulation plane to limit the shaft segments to pivoting along the articulation plane.

12. The steerable shaft for an interventional device as set forth in claim 11, wherein the at least one wire slot of each of the segments includes a pair of wire slots on diametrically opposite sides of the shaft segment to define a pair of wire channels each comprised of a plurality of the aligned wire slots, and wherein the at least one flat wire includes a pair of flat wires each received by one of the wire channels.

13. The steerable shaft for an interventional device as set forth in claim 11, wherein at least one axial end of each of the shaft segments includes a protrusion portion located in alignment with the at least one wire slot to define a pivot arc surface for an adjacent one of the shaft segments to pivot about along the articulation plane to establish a pivot angle during deflection of the steerable shaft.

14. The steerable shaft for an interventional device as set forth in claim 13, wherein the protrusion portion of each of the shaft segments slopes axially toward and engages the protrusion portion of another of the shaft segments such that the protrusion portions are pivotable about one another.

15. The steerable shaft for an interventional device as set forth in claim 13, wherein the plurality of shaft segments are comprised of a plurality of pairs of shaft segments that are integrally connected to one another along an edge and folded over one another along the edge with the protrusion portions of each pair pointed axially away from one another.

16. The steerable shaft for an interventional device as set forth in claim 1, wherein each of the shaft segments is formed from a sheet of a metal material.

17. The steerable shaft for an interventional device as set forth in claim 1, wherein at least one axial end of each of the shaft segments includes a protrusion portion to define a pivot arc surface for an adjacent one of the shaft segments to pivot about along the articulation plane to establish a pivot angle during deflection of the steerable shaft.

18. The steerable endoscope as set forth in claim 1, wherein each of the shaft segments defines a plurality of slots that each extend transversely to the axis in axially spaced relationship with one another to define at least one rib between the slots;
wherein at least one of the ribs of the shaft segments is bent radially inwardly to define the plurality of wire lumens along the slots between the ribs such that the steerable shaft is pivotable along the slots during tensioning of the at least one control wire; and
wherein the spine element includes an integral connection between each of the shaft segments circumferentially out of alignment with the slots and the ribs, and wherein the integral connection is of a stiff material for inhibiting pivoting of the steerable shaft between the shaft segments transversely to the articulation plane in order to limit pivoting of the steerable shaft to the articulation plane.

19. The steerable shaft for an interventional device as set forth in claim 18, wherein each of the shaft segments defines a first set of slots and a second set of the slots on diametrically opposite sides of the shaft segment to define two sets of the wire lumens on circumferentially opposite sides of the shaft segment, and wherein the at least one control wire includes a pair of control wires each received by one of the sets of wire lumens.

20. The steerable shaft for an interventional device as set forth in claim 19, wherein the first control wire and the second control wire are connected to one of the shaft sections at the second shaft end.
